(19) United States
(12) Patent Application Publication
(10) Pub. No.: US 2026/0087656 A1
(43) Pub. Date: Mar. 26, 2026

(54) ALIGNMENT BETWEEN INTRAVASCULAR IMAGES AND EXTRAVASCULAR IMAGES OF CARDIAC VASCULATURE

(71) Applicant: Boston Scientific Scimed, Inc., Maple Grove, MN (US)

(72) Inventors: Yan Li, Plymouth, MN (US); Kevin Bloms, Minnespolis, MN (US); Wenguang Li, Los Gatos, CA (US)

(73) Assignee: Boston Scientific Scimed, Inc., Maple Grove, MN (US)

(21) Appl. No.: 19/338,753

(22) Filed: Sep. 24, 2025

Related U.S. Application Data
(60) Provisional application No. 63/699,642, filed on Sep. 26, 2024.

(51) Int. Cl. G06T 7/33; A61B 5/00; G06T 3/14; G06T 3/60; G06T 7/73

(52) U.S. Cl. CPC G06T 7/337; A61B 5/742; G06T 3/14; G06T 3/60; G06T 7/74; G06T 2200/24; G06T 2207/10081; G06T 2207/10088; G06T 2207/10101; G06T 2207/10132; G06T 2207/20081; G06T 2207/30048; G06T 2207/30101

(57) ABSTRACT

The present disclosure provides to process a series of intravascular images, such as intravascular ultrasound (IVUS) images, to align the frames of the series of images longitudinally and angularly with respect to an extravascular image. In some examples, vessel fiducials are identified in each image modality and longitudinal and angular offsets are identified based on the location and angular orientation of the vessel fiducials. An aligned series of images can be generated by shifting and rotating frames of the series of images based on the identified offsets.

### CROSS-REFERENCE TO RELATED APPLICATIONS

This patent application claims the benefit of priority under 35 U.S.C. § 119 to U.S. Provisional Ser. No. 63/699,642, filed Sep. 26, 2024, which is herein incorporated by reference in its entirety.

### TECHNICAL FIELD

The present disclosure generally relates to aligning intravascular and extravascular images of cardiac vasculature both longitudinally and angularly. Particularly, but not exclusively, the present disclosure relates to aligning frames of a series of intravascular images, such as intravascular ultrasound (IVUS) images, with an extravascular image, such as an angiogram.

### BACKGROUND

Complementary imaging modalities are often relied on to diagnose and assess coronary artery diseases, such as, blocked blood vessels. Coronary angiography and IVUS are two such complementary imaging modalities. It is to be appreciated that angiography images provide a two-dimensional roadmap of the coronary arteries while IVUS images offer high-resolution cross-sectional images from within the vessel walls.

Although conventional tools allow for viewing these types of complimentary images, they lack the ability to correlate the images in meaningful ways. It is to be appreciated that intravascular images are often agnostic to the viewing angle. For example, IVUS images are captured as an ultrasound transducer is rotated within the vessel. As such, the actual viewing angle between frames can vary. Further, the viewing angle of an external image can also vary (e.g., based on the position of the patient with respect to the image acquisition system, or the like). As such, the viewing perspective between intravascular and extravascular images will not typically align.

The present disclosure addresses this issue by providing correlation between complementary coronary image modalities.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides to align complementary images of coronary vasculature, such as an angiogram and IVUS images. In particular, the disclosure provides to align both longitudinally and angularly, the IVUS image frames with the angiogram. This provides a significant advantage over conventional tools in that the spatial relationship between the vessel lumen and its surrounding structures is more clearly assessed as the images captured via different modalities are aligned and the imaged structures (e.g., lumen, border, etc.) are correlated with each other.

In some embodiments, the disclosure can be implemented as a complementary image modality correlation and visualization system. The system can comprise a processor; and memory comprising instructions executable by the processor, which when executed cause the system to receive an extravascular image of a vessel of a patient; receive a series of intravascular images of the vessel of the patient, the series of intravascular images comprising a plurality of frames; identify a plurality of vessel fiducials represented in the extravascular image and the series of intravascular images; determine an angular offset for at least one or more of the plurality of frames based in part on an angle of orientation of the plurality of vessel fiducials in the extravascular image and the series of intravascular images; and generate an aligned series of intravascular images comprising the plurality of frames, wherein the one or more of the plurality of frames are rotated based on the angular offset in the aligned series of intravascular images.

With some embodiments of the complementary image modality correlation and visualization system, the instructions when executed by the processor further cause the system to determine a longitudinal offset for at least one of the one or more of the plurality of frames based in part on a location of the plurality of vessel fiducials in the extravascular image and the series of intravascular images, wherein the at least one of the one or more of the plurality of frames is shifted longitudinally based on the longitudinal offset in the aligned series of intravascular images, and

With some embodiments of the complementary image modality correlation and visualization system, the instructions when executed by the processor further cause the system to generate a graphical user interface (GUI), the GUI comprising visual depictions of the extravascular image and the aligned series of intravascular images.

With some embodiments of the complementary image modality correlation and visualization system, the instructions when executed by the processor further cause the system to execute a machine learning (ML) model to infer the plurality of vessel fiducials from the extravascular image.

With some embodiments of the complementary image modality correlation and visualization system, the ML model is trained to infer locations and angle of orientation of the plurality of vessel fiducials from extravascular images.

With some embodiments of the complementary image modality correlation and visualization system, the ML model is a first ML model, and the instructions when executed by the processor further cause the system to execute a second ML model to infer frames of the series of intravascular images comprising the plurality of vessel fiducials from the series of intravascular images.

With some embodiments of the complementary image modality correlation and visualization system, the second ML model is trained to infer angle of orientation of vessel fiducials from a series of intravascular images.

With some embodiments of the complementary image modality correlation and visualization system, the plurality of vessel fiducials comprises a lumen geometry, a vessel geometry, a side branch location, a calcium morphology, a plaque distribution, a guide catheter, a thrombus, and/or a myocardium.

With some embodiments of the complementary image modality correlation and visualization system, the instructions when executed by the processor further cause the system to determine a mapping between the plurality of vessel fiducials represented in the extravascular image and the series of intravascular images.

With some embodiments of the complementary image modality correlation and visualization system, the instructions when executed by the processor further cause the system to determine a first angle, the first angle corresponding to an angle of orientation of a one of the plurality of vessel fiducials represented in the second of the plurality of frames; determine a second angle corresponding to an angle of orientation of the one of the plurality of vessel fiducials represented in the extravascular image; and derive an offset between the first angle and the second angle.

With some embodiments of the complementary image modality correlation and visualization system, the instructions when executed by the processor further cause the system to co-register the plurality of frames of the series of intravascular images with the extravascular image based in part on the mapping between the plurality of vessel fiducials; and rotate the co-registered plurality of frames of the series of intravascular images based in part on the derived offset between the first angle and the second angle.

With some embodiments of the complementary image modality correlation and visualization system, the instructions when executed by the processor further cause the system to generate a curve comprising indications of the longitudinal offset and/or the angular offset for the plurality of frames based on a line fitting algorithm applied to the longitudinal offset and/or the angular offset.

With some embodiments of the complementary image modality correlation and visualization system, the series of intravascular images are intravascular ultrasound (IVUS) images or optical coherence tomography (OCT) images.

With some embodiments of the complementary image modality correlation and visualization system, the extravascular image is an angiographic image, a computed tomography (CT) image, or a magnetic resonance image (MRI).

In some embodiments, the disclosure can be implemented as a non-transitory machine readable storage device. The storage device can comprise a plurality of instructions that in response to being executed by a processor of a complementary image modality correlation and visualization system cause the processor to receive an extravascular image of a vessel of a patient; receive a series of intravascular images of the vessel of the patient, the series of intravascular images comprising a plurality of frames; identify a plurality of vessel fiducials represented in the extravascular image and the series of intravascular images; determine an angular offset for at least one or more of the plurality of frames based in part on an angle of orientation of the plurality of vessel fiducials in the extravascular image and the series of intravascular images; and generate an aligned series of intravascular images comprising the plurality of frames, wherein the one or more of the plurality of frames are rotated based on the angular offset in the aligned series of intravascular images.

With some embodiments of the storage device, the instructions when executed by the processor further cause the processor to determine a longitudinal offset for at least one of the one or more of the plurality of frames based in part on a location of the plurality of vessel fiducials in the extravascular image and the series of intravascular images, wherein the at least one or more of the plurality of frames is shifted longitudinally based on the longitudinal offset in the aligned series of intravascular images.

With some embodiments of the storage device, the instructions when executed by the processor further cause the processor to generate a graphical user interface (GUI), the GUI comprising visual depictions of the extravascular image and the aligned series of intravascular images.

With some embodiments of the storage device, the instructions when executed by the processor further cause the processor to execute a first machine learning (ML) model to infer the plurality of vessel fiducials from the extravascular image; and execute a second ML model to infer frames of the series of intravascular images comprising the plurality of vessel fiducials from the series of intravascular images.

With some embodiments of the storage device, the first ML model is trained to infer locations and angle of orientation of the plurality of vessel fiducials from extravascular images, and the second ML model is trained to infer an angle of orientation of vessel fiducials from a series of intravascular images

In some embodiments, the disclosure can be implemented as a method for a complementary image modality correlation and visualization system. The method can comprise receiving, by a processor, an extravascular image of a vessel of a patient; receiving, by the processor, a series of intravascular images of the vessel of the patient, the series of intravascular images comprising a plurality of frames; identifying, by the processor, a plurality of vessel fiducials represented in the extravascular image and the series of intravascular images; determining, by the processor, an angular offset for at least one or more of the plurality of frames based in part on an angle of orientation of the plurality of vessel fiducials in the extravascular image and the series of intravascular images; and generating, by the processor, an aligned series of intravascular images comprising the plurality of frames, wherein the one or more of the plurality of frames is rotated based on the angular offset in the aligned series of intravascular images.

With some embodiments, the method can further comprise determining, by the processor, a longitudinal offset for at least one of the one or more of the plurality of frames based in part on a location of the plurality of vessel fiducials in the extravascular image and the series of intravascular images, wherein the at least one of the one or more of the plurality of frames is shifted longitudinally based on the longitudinal offset in the aligned series of intravascular images.

With some embodiments, the method can further comprise generating, by the processor, a graphical user interface (GUI), the GUI comprising visual depictions of the extravascular image and the aligned series of intravascular images.

With some embodiments of the method, identifying the plurality of vessel fiducials represented in the extravascular image comprises executing a machine learning (ML) model to infer the plurality of vessel fiducials from the extravascular image.

With some embodiments of the method, the ML model is trained to infer locations and angle of orientation of the plurality of vessel fiducials from extravascular images.

With some embodiments of the method, the ML model is a first ML model, and identifying the plurality of vessel fiducials represented in the series of intravascular images comprises executing a second ML model to infer frames of the series of intravascular images comprising the plurality of vessel fiducials from the series of intravascular images.

With some embodiments of the method, the second ML model is trained to angle of orientation of vessel fiducials from a series of intravascular images.

With some embodiments of the method, the plurality of vessel fiducials comprises a lumen geometry, a vessel geometry, a side branch location, a calcium morphology, a plaque distribution, a guide catheter, a thrombus, and/or a myocardium.

With some embodiments of the method, determining the longitudinal offset for at least the first one of the plurality of frames based in part on the location of the plurality of vessel fiducials in the extravascular image and the series of intravascular images comprises determining, by the processor, a mapping between the plurality of vessel fiducials represented in the extravascular image and the series of intravascular images.

With some embodiments of the method, determining the angular offset for at least the second one of the plurality of frames based in part on the angle of orientation of the plurality of vessel fiducials in the extravascular image and the series of intravascular images comprises determining, by the processor, a first angle, the first angle corresponding to an angle of orientation of a one of the plurality of vessel fiducials represented in the second of the plurality of frames; determining, by the processor, a second angle corresponding to an angle of orientation of the one of the plurality of vessel fiducials represented in the extravascular image; and deriving, by the processor, an offset between the first angle and the second angle.

With some embodiments of the method, generating the aligned series of intravascular images comprises co-registering, by the processor, the plurality of frames of the series of intravascular images with the extravascular image based in part on the mapping between the plurality of vessel fiducials; and rotating, by the processor, the co-registered plurality of frames of the series of intravascular images based in part on the derived offset between the first angle and the second angle.

With some embodiments of the method, determining the longitudinal offset for at least the first one of the plurality of frames based in part on the location of the plurality of vessel fiducials in the extravascular image and the series of intravascular images and/or determining the angular offset for at least the second one of the plurality of frames based in part on the angle of orientation of the plurality of vessel fiducials in the extravascular image and the series of intravascular images comprises generating a curve comprising indications of the longitudinal offset and/or the angular offset for the plurality of frames based on a line fitting algorithm applied to the longitudinal offset and/or the angular offset.

With some embodiments of the method, the series of intravascular images are intravascular ultrasound (IVUS) images or optical coherence tomography (OCT) images.

With some embodiments of the method, the extravascular image is an angiographic image, a computed tomography (CT) image, or a magnetic resonance image (MRI).

### BRIEF DESCRIPTION OF THE DRAWINGS

To easily identify the discussion of any element or act, the most significant digit or digits in a reference number refer to the figure number in which that element is first introduced.

FIG. 1 illustrates a complementary image modality correlation and visualization system configured to longitudinally and angularly align a series of intravascular images with an extravascular image.

FIG. 2 illustrates an example extravascular image.

FIG. 3A and FIG. 3B illustrate an example series of intravascular images.

FIG. 4 illustrates a logic flow to longitudinally and angularly align intravascular images with an extravascular image.

FIG. 5A illustrates vessel fiducials identified from the extravascular image of FIG. 2 and frames of the series of intravascular images of FIG. 3A and FIG. 3B.

FIG. 5B illustrates a plot showing longitudinal offsets for the series of intravascular images of FIG. 3A and FIG. 3B based on the vessel fiducials.

FIG. 5C illustrates a shifted series of intravascular images.

FIG. 6A illustrates a plot showing angular offsets for the shifted series of intravascular images of FIG. 5C based on the vessel fiducials.

FIG. 6B illustrates a rotated series of intravascular images.

FIG. 7 illustrates an example graphical user interface (GUI) to display visual representations of the extravascular image along with visual representations of the series of intravascular images aligned with the viewing perspective of the extravascular image.

FIG. 8 illustrates an exemplary artificial intelligence/machine learning (AI/ML) system suitable for use to train ML models.

FIG. 9 illustrates a computer-readable storage medium.

FIG. 10 illustrates a diagrammatic representation of a machine in the form of a computer system within which a set of instructions may be executed for causing the machine to perform any one or more of the methodologies discussed herein, according to an example embodiment.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to intravascular images and extravascular images. The following disclosure uses IVUS and angiography as example imaging modalities. However, other imaging modalities could be used in place of the described image modalities (e.g., optical coherence tomography (OCT), magnetic resonance imaging (MRI), or the like).

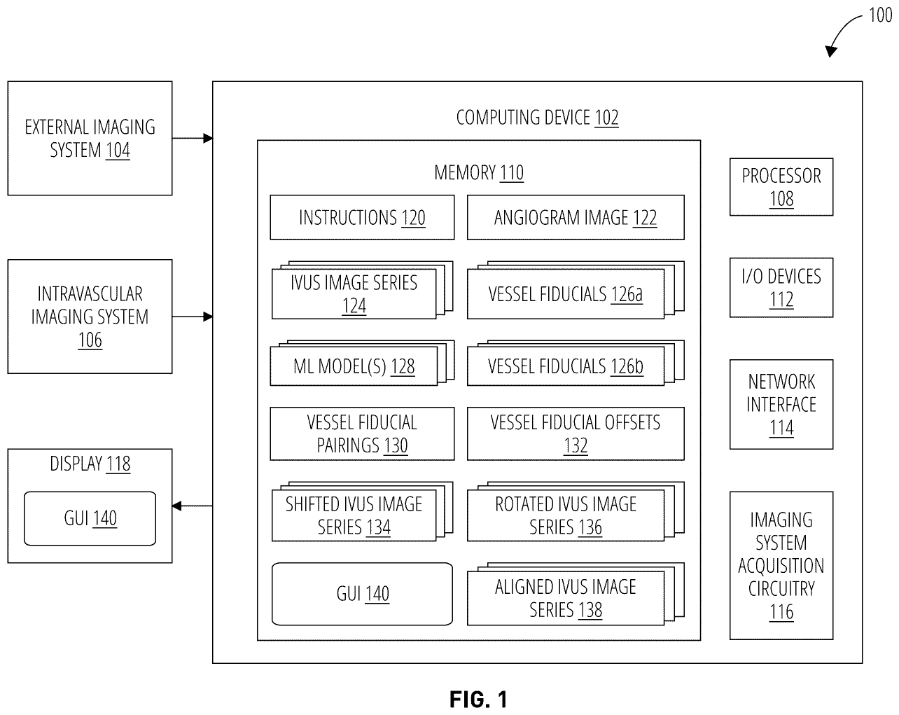

FIG. 1 illustrates a complementary image modality correlation and visualization system 100, according to some embodiments of the present disclosure. In general, complementary image modality correlation and visualization system 100 is a system for processing, correlating, and presenting a series of intravascular images (e.g., IVUS image frames) with an extravascular image (e.g., angiogram image) of the same cardiac vessel. With some embodiments, complementary image modality correlation and visualization system 100 can be implemented in a commercial IVUS guidance and/or navigation system, such as, for example, the AVVIGO™ Guidance System available from Boston Scientific®. The present disclosure provides advantages over prior or conventional IVUS navigation systems by longitudinally and angularly aligning the IVUS image frames with the angiogram image.

The combination of angular and longitudinal alignment between IVUS images and an angiographic image is significant in that it provides consistency of the views and facilitates a more intuitive interpretation of IVUS images. For example, rotating the myocardium to the bottom of the frame in IVUS images to correlate with the view in the angiographic image provides spatial context. Further, the myocardium can then serve as a reference point for lesion location and depth within the vessel wall. As such, aligning it with the bottom of the image frames ensures consistency between views of the different image modalities and can facilitate better assessment of cardiac artery disease (e.g., lesion depth, plaque burden, vessel remodeling, etc.) by clinicians.

With some embodiments, the complementary image modality correlation and visualization system 100 could be implemented as part of an angiogram imager (e.g., a c-arm imager, or the like). The complementary image modality correlation and visualization system 100 includes a computing device 102. Optionally, complementary image modality correlation and visualization system 100 includes external imaging system 104, and/or intravascular imaging system 106.

Computing device 102 can be any of a variety of computing devices. In some embodiments, computing device 102 can be incorporated into and/or implemented by a console to be coupled to an intravascular imaging device (e.g., an IVUS catheter, or the like). With some embodiments, computing device 102 can be a workstation or server communicatively coupled to external imaging system 104 and/or intravascular imaging system 106. With still other embodiments, computing device 102 can be provided by a cloud based computing device, such as, by a computing as a service system accessibly over a network (e.g., the Internet, an intranet, a wide area network, or the like). Computing device 102 can include processor 108, memory 110, I/O devices 112, network interface 114, imaging system acquisition circuitry 116, and display 118.

The processor 108 may include circuity or processor logic, such as, for example, any of a variety of commercial processors. In some examples, processor 108 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 108 may include graphics processing portions and may include dedicated memory, multiple-threaded processing and/or some other parallel processing capability. In some examples, the processor 108 may be an application specific integrated circuit (ASIC) or a field programmable integrated circuit (FPGA).

The memory 110 may include logic, a portion of which includes arrays of integrated circuits, forming non-volatile memory to persistently store data or a combination of non-volatile memory and volatile memory. It is to be appreciated, that the memory 110 may be based on any of a variety of technologies. In particular, the arrays of integrated circuits included in memory 110 may be arranged to form one or more types of memory, such as, for example, dynamic random access memory (DRAM), NAND memory, NOR memory, or the like.

I/O devices 112 can be any of a variety of devices to receive input and/or provide output. For example, I/O devices 112 can include, a keyboard, a mouse, a joystick, a foot pedal, a display, a touch enabled display, a haptic feedback device, an LED, or the like.

Network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards. For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, 5G, or the like.

The imaging system acquisition circuitry 116 may include circuity including custom manufactured or specially programmed circuitry configured to receive or receive and send signals between external imaging system 104 and/or intravascular imaging system 106 including indications of images, image frames, or a series of images.

Display 118 may include any of a variety of devices arranged to display graphical information, such as, a light emitting diode (LED) display, or the like). It is to be appreciated that although display 118 is depicted separate from computing device 102, display 118 could be implemented as part of computing device 102 or be distinct from computing device 102.

Memory 110 can include instructions 120, angiogram image 122, IVUS image series 124, vessel fiducials 126a, vessel fiducials 126b, ML model(s) 128, vessel fiducial pairings 130, vessel fiducial offsets 132, rotated IVUS image series 136, aligned IVUS image series 138, and GUI 140.

In general, during operation, processor 108 can execute instructions 120 to cause computing device 102 to generate rotated IVUS image series 136 from IVUS image series 124 to align (longitudinally and angularly) the frames in IVUS image series 124 with the viewing perspective of angiogram image 122.

With some examples, processor 108 can execute instructions 120 to receive (e.g., from external imaging system 104, or the like) angiogram image 122 of a cardiac vessel. In some examples, angiogram image 122 can be an angiogram video (e.g., a cine-loop, or the like) while in other examples, angiogram image 122 can be a single frame. Where angiogram image 122 comprises a video (e.g., a series of frames, or the like) processor 108 can execute instructions 120 to select one of the frames of the video as angiogram image 122.

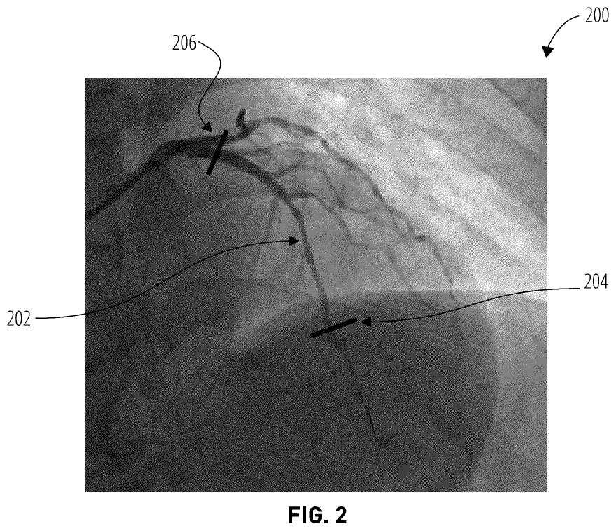

As an example, FIG. 2 illustrates an extravascular image 200 of a vessel 202 of a patient, in the form of an angiographic image. It is to be appreciated that extravascular image 200 can be captured via any conventional extravascular image modality and equipment. With some embodiments, where complementary image modality correlation and visualization system 100 includes external imaging system 104, external imaging system 104 can be configured to capture angiogram image 122 (e.g., like extravascular image 200) and processor 108 can execute instructions 120 to receive angiogram image 122 (e.g., as a data structure, in DICOM image format, or the like). In other embodiments, where external imaging system 104 is not part of complementary image modality correlation and visualization system 100, processor 108 can be configured to execute instructions 120 to receive angiogram image 122 from another computing device (not shown).

Further, processor 108 can execute instructions 120 to receive (e.g., from intravascular imaging system 106, or the like) IVUS image series 124 of the cardiac vessel represented in angiogram image 122. In general, IVUS image series 124 comprises a series of IVUS image frames where the frames are captured by an IVUS imaging catheter while the catheter is being pulled back from a distal point in the vessel to a proximal point in the vessel. It is to be appreciated that the IVUS image series 124 includes multiple frames, which when represented co-linearly can be used to form an image of the vessel.

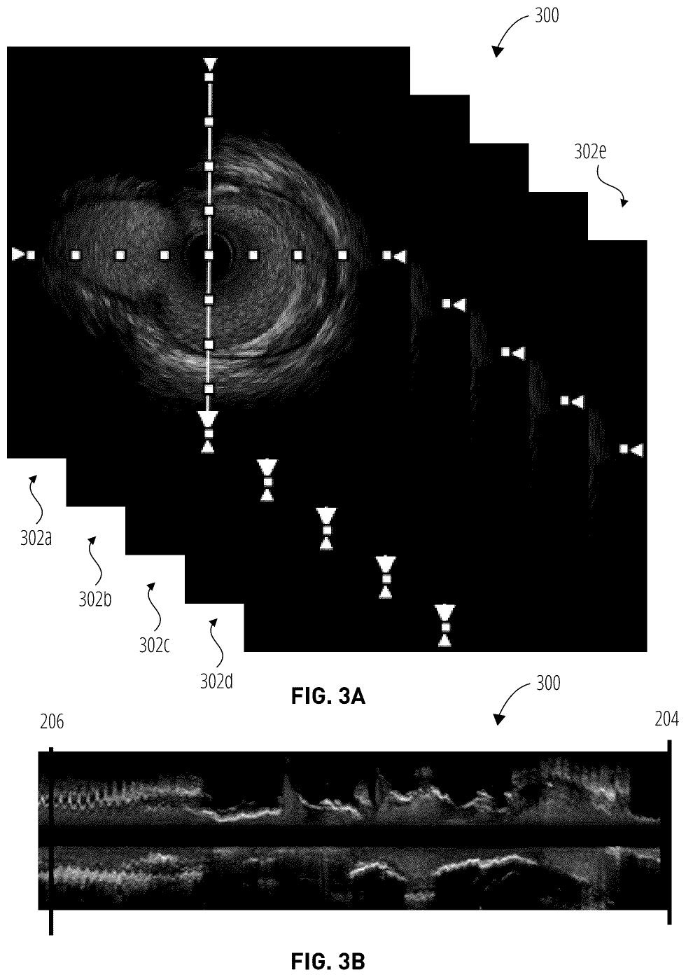

For example, FIG. 3A illustrates IVUS image series 300 comprising IVUS image frames 302a, 302b, 302c, 302d, and 302e. It is to be appreciated that IVUS image series 300 can be captured via any conventional intravascular image modality. Further, IVUS image series 300 can comprise any number of image frames. The five (5) frames depicted here are shown for purposes of clarity only. IVUS image series 300 can be captured via an imaging catheter while the imaging catheter is pulled back through vessel 202 from a distal end 204 to a proximal end 206 (FIG. 2 and FIG. 3B). Additionally, IVUS image series (e.g., IVUS image frames 302a, etc.) can be and represented as a longitudinal slice of the vessel 202, for example, as shown in FIG. 3B.

With some embodiments, where complementary image modality correlation and visualization system 100 includes intravascular imaging system 106, intravascular imaging system 106 can be configured to capture IVUS image series 124 (e.g., like IVUS image series 300) and processor 108 can execute instructions 120 to receive IVUS image series 124 (e.g., as a data structure, in DICOM image format, or the like). In other embodiments, where intravascular imaging system 106 is not part of complementary image modality correlation and visualization system 100, processor 108 can be configured to execute instructions 120 to receive IVUS image series 124 from another computing device (not shown).

In either case where external imaging system 104 and/or intravascular imaging system 106 is part of complementary image modality correlation and visualization system 100, imaging system acquisition circuitry 116 can be configured to communicate electronic signals with external imaging system 104 and/or intravascular imaging system 106 to receive angiogram image 122 and/or IVUS image series 124, respectively.

As noted above, given angiogram image 122 and IVUS image series 124, complementary image modality correlation and visualization system 100 can be configured to align IVUS image series 124 with the viewing perspective of angiogram image 122 based on vessel fiducials 126a. Processor 108 can execute instructions 120 to align the frames of IVUS image series 124 longitudinally and angularly to match the viewing perspective depicted in angiogram image 122. With some examples, processor 108 can execute instructions 120 to implement a longitudinal and angular alignment logic flow with angiogram image 122 and IVUS image series 124 as inputs.

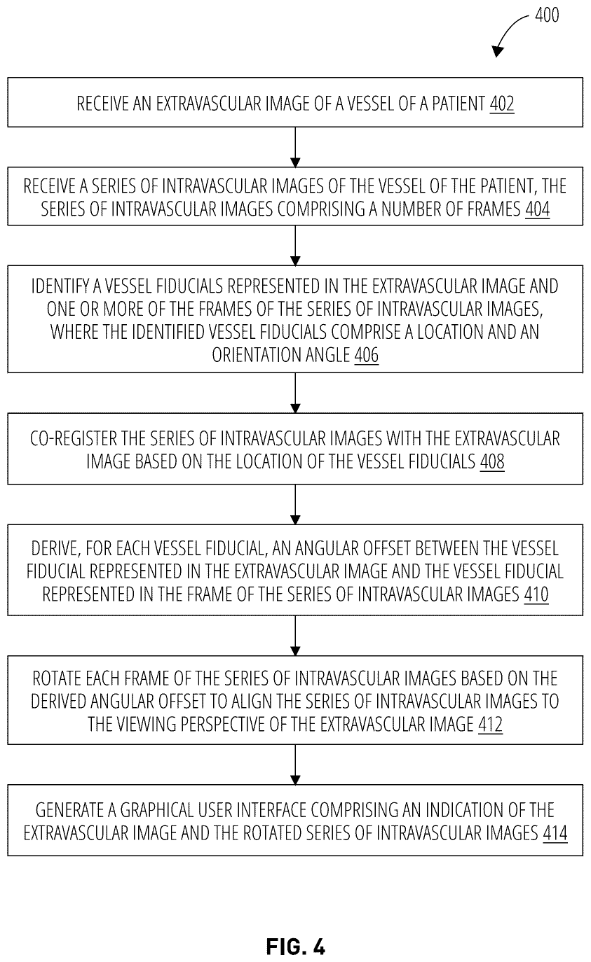

FIG. 4 illustrates a logic flow 400 to align a series of IVUS images with an angiographic image, according to embodiments of the present disclosure. The logic flow 400 can be implemented by the complementary image modality correlation and visualization system 100 and will be described with reference to complementary image modality correlation and visualization system 100 for clarity of presentation. However, it is noted that logic flow 400 could also be implemented by an extravascular imaging system, an IVUS guidance system, or another medical image viewing system different than complementary image modality correlation and visualization system 100.

Logic flow 400 can begin at block 402. At block 402 “receive an extravascular image of a vessel of a patient” an extravascular image of a vessel of a patient can be received. For example, processor 108 can execute instructions 120 to receive information elements comprising indications of angiogram image 122 from external imaging system 104 or from another computer device where the angiogram image 122 was captured previously.

Continuing to block 404 “receive a series of intravascular images of the vessel of the patient” a series of intravascular images can be received. For example, processor 108 can execute instructions 120 to receive information elements comprising indications of IVUS image series 124 from intravascular imaging system 106.

Continuing to block 406 “identify a vessel fiducials represented in the extravascular image and one or more of the frames of the series of intravascular images, where the identified vessel fiducials comprise a location and an orientation angle” vessel fiducials represented in the extravascular image and the frames of the series of intravascular images can be identified. For example, processor 108 can execute instructions 120 to identify (e.g., a vessel fiducials) side branches of the vessel 202 represented in angiogram image 122 and IVUS image series 124.

It is to be appreciated that a variety of techniques exist to identify the location of vessel side branches represented in both internal and external images. For example, side branch identification and matching are often used to co-register intravascular images to an extravascular image. In the present disclosure, processor 108 can execute instructions 120 to identify both the location of side branches and the angle of orientation of the side branches from angiogram image 122 and store an indication of the side branch location and angle in vessel fiducials 126a. In further embodiments, processor 108 can execute instructions 120 to identify the location and angle of orientation of other “markers” unique to vessel 202 represented in angiogram image 122 (e.g., myocardium, calcifications, thrombus, plaque morphology, or the like) and store indications of the location and angle of orientation in vessel fiducials 126a. In further embodiments, other medical devices present in the vessel 202, such as a guide catheter, can be used by processor 108 which can execute instructions 120 to identify vessel fiducials 126a based in part on information or signals received from or identified based on such other medical devices.

Similarly, processor 108 can execute instructions 120 to identify both the location of side branches and the angle of orientation of the side branches from IVUS image series 124 (or frames of IVUS image series 124) and store an indication of the side branch location and angle in vessel fiducials 126b. In further embodiments, processor 108 can execute instructions 120 to identify the location and angle of orientation of other “markers” unique to vessel 202 represented in frames of IVUS image series 124 (e.g., myocardium, calcifications, thrombus, plaque morphology, or the like) and store indications of the location and angle of orientation in vessel fiducials 126b. In further embodiments, other medical devices present in the vessel 202, such as a guide catheter, can be used by processor 108 which can execute instructions 120 to identify vessel fiducials 126b based in part on information or signals received from or identified based on such other medical devices.

With some embodiments, processor 108 can identify the vessel fiducials 126a and 126b using image processing techniques and/or machine learning (ML) inference. For example, processor 108 can execute instructions 120 to identify vessel fiducials 126a from angiogram image 122 and vessel fiducials 126b from IVUS image series 124 using ML model(s) 128, where ML model(s) 128 are trained to identify the location and angle of vessel fiducials (e.g., side branches, etc.) in extravascular or intravascular images. It is noted that a first one or more of ML model(s) 128 may be employed to infer vessel fiducials 126a from angiogram image 122 while a second one or more ML model(s) 128 may be employed to infer vessel fiducials 126b from IVUS image series 124.

With some embodiments, processor 108 can be configured to execute instructions 120 to identify vessel fiducials 126a and 126b from angiogram image 122 and IVUS image series 124, respectively, and to generate vessel fiducial pairings 130 based on a side-branch identification and/or matching process, such as, for example, the processes outlined in United States Provisional Ser. No. 63/588,546 filed Oct. 6, 2023 and titled “Side Branch Detection From Angiographic Images;” United States Provisional Ser. No. 63/588,552 filed Oct. 6, 2023 and titled “Automated Side Branch Detection and Angiographic Image Co-Registration;” and United States Provisional Ser. No. 63/588,571 filed Oct. 6, 2023 and titled “Cross-Modality Vascular Image Side Branch Matching;” which applications are incorporated by reference in their entirety.

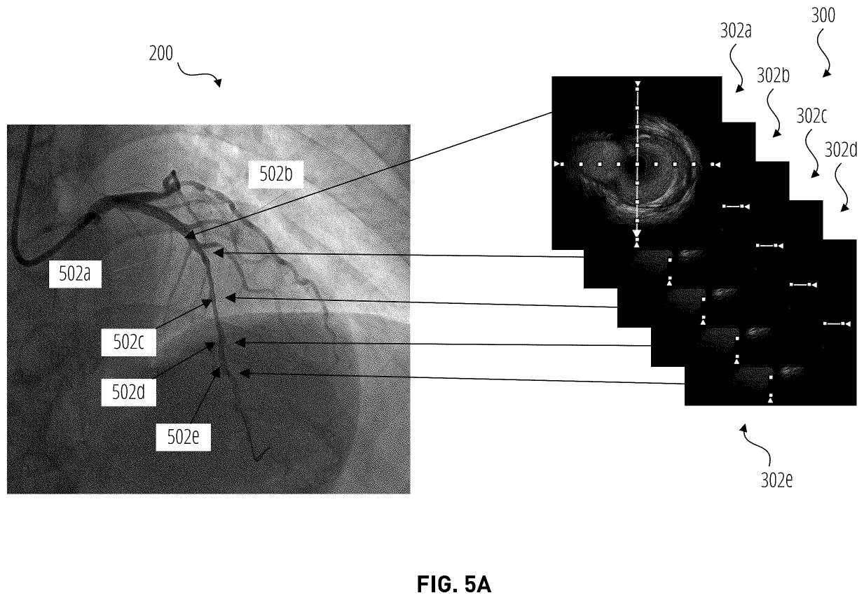

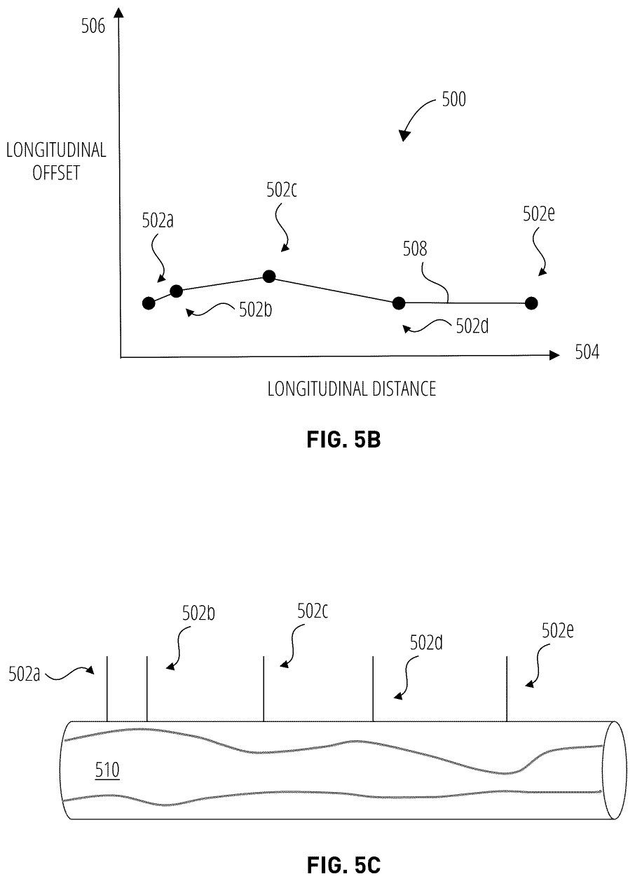

Turning briefly to FIG. 5A, extravascular image 200 is depicted with vessel fiducials 502a, 502b, 502c, 502d, and 502e identified. Processor 108 can execute instructions 120 to identify the location of the vessel fiducials 502a to 502e as well an angle of orientation. In some embodiments, the angle of the vessel fiducials 502a to 502e can be derived based on a baseline setting. For example, the baseline setting may be zero (0) degrees equals the Z direction from the two-dimensional (2D) image towards the viewer, or the like.

Returning to logic flow 400 of FIG. 4 and continuing to block 408 “co-register the series of intravascular images with the extravascular image based on the location of the vessel fiducials” the frames of the series of intravascular images can be mapped, or registered, to locations (e.g., x and y coordinates, or the like) on the extravascular image based on the vessel fiducials. For example, processor 108 can execute instructions 120 to determine locations on angiogram image 122 for each frame of IVUS image series 124 based on the vessel fiducials 126a and 126b and store indications of the locations in vessel fiducial pairings 130. Said differently, vessel fiducial pairings 130 can comprise indications of which vessel fiducials 126a identified from angiogram image 122 match, or pair with, which vessel fiducials 126b identified from IVUS image series 124.

Turning briefly again to FIG. 5A, assume that vessel fiducial 502a is identified in IVUS image frame 302a of IVUS image series 300, vessel fiducial 502b is identified in IVUS image frame 302b of IVUS image series 300, etc. In such an example scenario, processor 108 could execute instructions 120 to map the location of IVUS image frame 302a to the location on extravascular image 200 corresponding to the vessel fiducial 502a and to map the location of IVUS image frame 302b to the location on extravascular image 200 corresponding to the vessel fiducial 502b. Or said differently, processor 108 can execute instructions 120 to match the vessel fiducial 502a identified on extravascular image 200 with the vessel fiducial represented in IVUS image frame 302a and to match the vessel fiducial 502b identified on extravascular image 200 with the vessel fiducial represented in IVUS image frame 302b.

For example, each of the vessel fiducials of frames in IVUS image series 124 (e.g., vessel fiducials 126b) can be mapped to locations on angiogram image 122 based on vessel fiducials 126a. In some embodiments, processor 108 can execute instructions 120 to identify a longitudinal offset for frames of IVUS image series 124 based on vessel fiducials 126a and 126b and vessel fiducial pairings 130 and store indications of the longitudinal offset in vessel fiducial offsets 132. For example, FIG. 5B illustrates plot 500 plotting points of each vessel fiducials 502a to 502e at points along the longitudinal distance of IVUS image series 300 along the x axis 504 and longitudinal offset plotted on the y axis 506. In general, the longitudinal offset can be generated based on the locations of the vessel fiducials 502a to 502e on extravascular image 200 (e.g., locations of vessel fiducials 126a on angiogram image 122, or the like). Further, longitudinal offset curve 508 can be generated (e.g., based on one or more line fitting algorithms, or the like) and IVUS image frames 302a to 302e adjusted based on the longitudinal offset specified by longitudinal offset curve 508.

FIG. 5C illustrates shifted IVUS image series 510 (e.g., shifted IVUS image series 134, or the like) where the frames (e.g., IVUS image frames 302a to 302e have been shifted longitudinally based on the vessel fiducial offsets 132 derived based on the vessel fiducials. With some examples, the longitudinal registration is based on a centerline of the vessel 202. With some embodiments, processor 108 can be configured to execute instructions 120 to generate shifted IVUS image series 134 based on a co-registration process, such as, for example, the co-registration process outlined in United States Provisional Ser. No. 63/588,559 filed Oct. 6, 2023, and titled “Live Co-Registration of Extravascular and Intravascular Images,” which application is incorporated by reference in its entirety.

With some examples, processor 108 can execute instructions 120 to identify longitudinal offset curve 508 on a segment-by-segment basis. For example, a longitudinal offset for frames in a first segment (e.g., the segment of IVUS image series 300 proximal to vessel fiducial 502a, or the like) can be determined based on a first selection of co-registration methodologies disclosed herein while a longitudinal offset for frames in another segment (e.g., segment of IVUS image series 300 between vessel fiducial 502a and vessel fiducial 502b, or the like) can be determined based on a second selection of co-registration methodologies disclosed herein.

Returning to logic flow 400 of FIG. 4. With some embodiments, block 408 can be omitted from the logic flow 400. For example, the complementary image modality correlation and visualization system 100 can be configured to generate aligned IVUS image series based just on the rotated IVUS image series 136. Said differently, processor 108 can execute instructions 120 to generate aligned IVUS image series 138 based just on angular offsets described herein. In such embodiments, logic flow 400 can proceed directly from block 406 to block 410. Further, in such embodiments, vessel fiducial offsets 132 will comprise indications of the angular offsets and may not comprise indications of longitudinal offsets. At block 410 “derive, for each vessel fiducial, an angular offset between the vessel fiducial represented in the extravascular image and the vessel fiducial represented in the frame of the series of intravascular images” angular offsets between each paired vessel fiducial can be derived. For example, processor 108 can execute instructions 120 to identify the angular offset for each fiducial of vessel fiducials 126a and 126b and derive an offset between angles for each pair of matched vessel fiducials. With some examples, processor 108 can execute instructions 120 to derive the offset between angles based on linear mapping algorithms and store an indication of the derived angle offsets as vessel fiducial offsets 132.

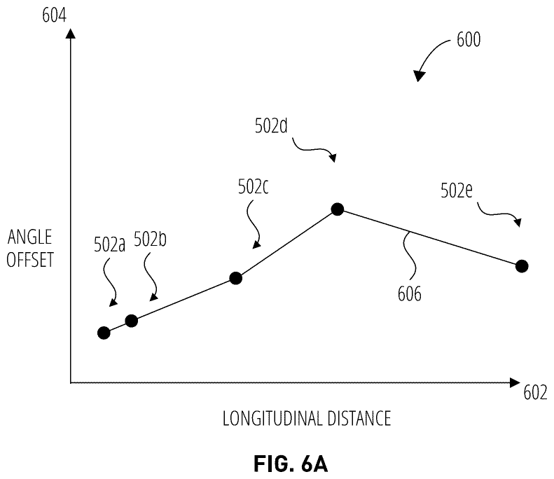

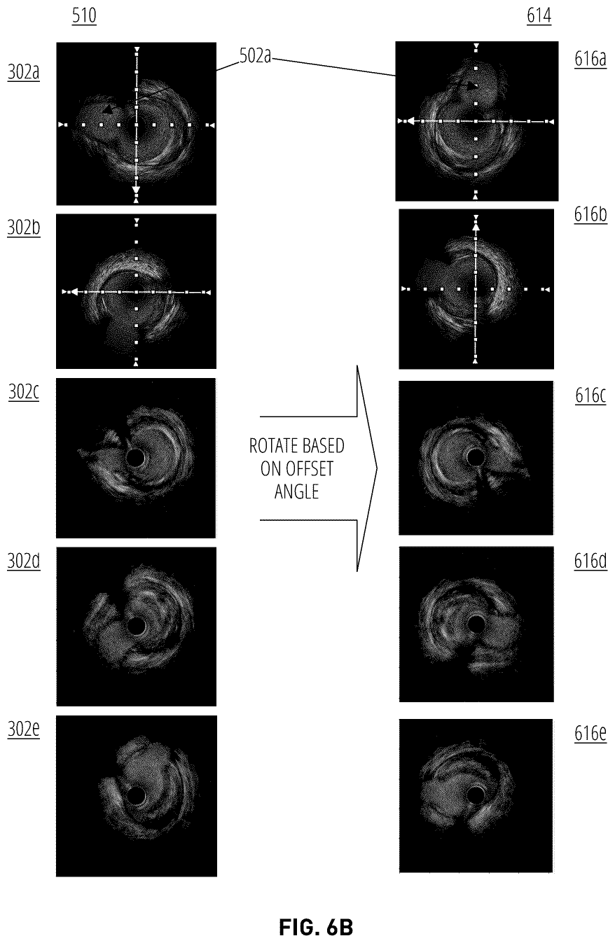

For example, FIG. 6A illustrates plot 600 showing points representing each angle offset between vessel fiducials 502a to 502e plotted on the y axis 604 at points along the longitudinal distance of shifted IVUS image series 510 plotted on the x axis 602. From these points, angle offset curve 606 can be generated representing angle offsets for each frame of shifted IVUS image series 510 (e.g., IVUS image frames 302a to 302e, or the like). As noted above, the angle offset 606 can be generated linearly and/or based on one or more line fitting or line smoothing algorithms.

With some examples, processor 108 can execute instructions 120 to identify angle offset 606 on a segment-by-segment basis. For example, an alignment offset for frames in a first segment (e.g., the segment of IVUS image series 300 proximal to vessel fiducial 502a, or the like) can be determined based on a first selection of alignment methodologies disclosed herein while an alignment offset for frames in another segment (e.g., segment of IVUS image series 300 between vessel fiducial 502a and vessel fiducial 502b, or the like) can be determined based on a second selection of alignment methodologies disclosed herein.

Returning to logic flow 400 of FIG. 4 and continuing to block 412 “rotate each frame of the series of intravascular images based on the derived angular offset to align the series of intravascular images to the viewing perspective of the extravascular image” frames of the series of intravascular images can be rotated based on the derived angle offsets to align the series of intravascular images with the viewing perspective of the extravascular image. For example, processor 108 can execute instructions 120 to rotate frames of IVUS image series 124 based on vessel fiducial offsets 132 such that IVUS image series 124 is aligned both longitudinally and angularly with the viewing perspective of angiogram image 122 and store the rotated frames as rotated IVUS image series 136.

FIG. 6B illustrates IVUS image frames 302a to 302e of shifted IVUS image series 510 rotated into IVUS image frames 616a to 616e, respectively. IVUS image frames 616a to 616e can form rotated IVUS image series 614 corresponding to shifted IVUS image series 510 aligned with extravascular image 200 as outlined above (e.g., based on longitudinal and angular alignment between vessel fiducials 502a to 502e). For example, IVUS image frame 302a can be rotated based on an offset between the angle of vessel fiducial 502a represented in IVUS image frame 302a and the angle of vessel fiducial 502a represented in extravascular image 200 to map, or correlate, the viewing angle of IVUS image frame 302a with that of extravascular image 200. In some embodiments, each of IVUS image frames 302a, 302b, 302c, 302d, and 302e can be rotated to form IVUS image frames 616a, 616b, 616c, 616d, and 616e, respectively based on the derived offset angle (e.g., angle offset curve 606, or the like).

Accordingly, as outlined above, processor 108 can execute instructions 120 to longitudinally align frames within a series of intravascular images (e.g., IVUS image series 124) with a viewing perspective of an external image (e.g., angiogram image 122) to form shifted IVUS image series 134 and then angularly align frames within the shifted IVUS image series 134 to form rotated IVUS image series 136. Processor 108 can execute instructions 120 to generate aligned IVUS image series 138 from shifted IVUS image series 134 and rotated IVUS image series 136 such that the perspective in which the vessel structure is viewed in aligned IVUS image series 138 aligns with the viewed perspective in angiogram image 122.

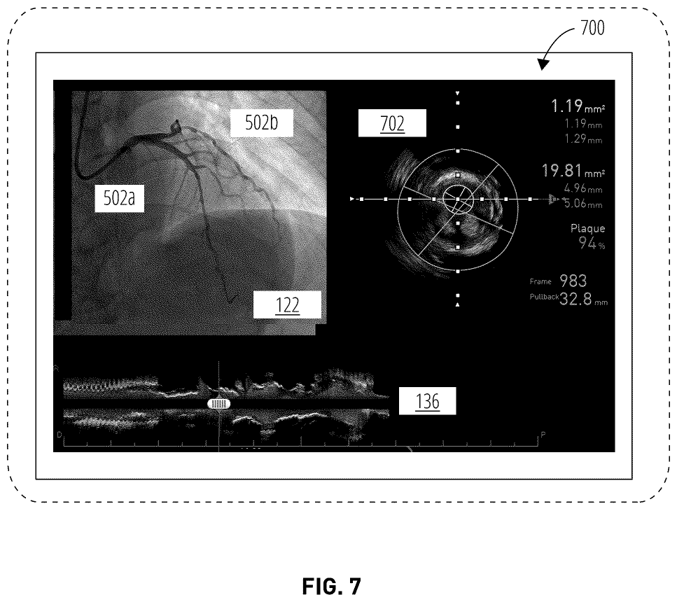

Returning to logic flow 400 of FIG. 4 and continuing to block 414 “generate a graphical user interface comprising an indication of the extravascular image and the rotated series of intravascular images” a graphical user interface (GUI) comprising an indication of the extravascular image, and the rotated intravascular images can be generated. As such, a visual representation of frames from a series of intravascular images (e.g., IVUS image series 124) longitudinally and angularly aligned with a vessel as viewed in an extravascular image can be presented to a user. For example, processor 108 can execute instructions 120 to generate GUI 140 comprising visual indications of angiogram image 122 and rotated IVUS image series 136. As a specific example, processor 108 can execute instructions 120 to generate GUI 700 (FIG. 7) as GUI 140 and cause GUI 700 to be displayed on display 118.

FIG. 7 illustrates an example GUI 700, which can be generated in accordance with some embodiments of the present disclosure. As noted, GUI 700 can be GUI 140 of FIG. 1. For example, processor 108 can execute instructions 120 to generate GUI 140 having graphical components and an arrangement as depicted in GUI 700 of FIG. 7. GUI 700 can include graphical indications of angiogram image 122 and rotated IVUS image series 136 as well as on-axis view 702 showing a frame of rotated IVUS image series 136.

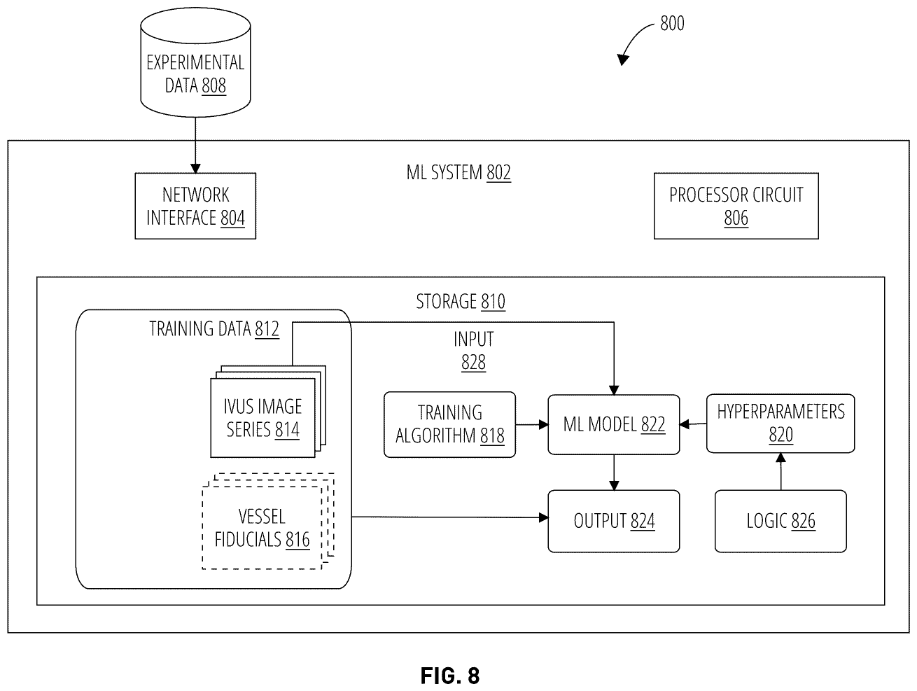

As noted, with some embodiments, processor 108 of computing device 102 can execute instructions 120 to generate vessel fiducials 126a, vessel fiducials 126b, vessel fiducial pairings 130, and/or vessel fiducial offsets 132 using an ML model. In such examples, the ML model can be stored in memory 110 of computing device 102. It will be appreciated, that prior to being deployed, the ML model is to be trained. FIG. 8 illustrates an ML environment 800, which can be used to train an ML model that may later be used to generate (or infer) a mapping or vessel fiducials as outlined herein. The ML environment 800 may include an ML system 802, such as a computing device that applies an ML algorithm to learn relationships between an input and an inferred output. In this example, the ML algorithm can learn relationships between an input (e.g., IVUS image series) and an output (e.g., vessel fiducials). It is noted that the ML environment 800 could be implemented to learn relationships between other inputs (e.g., angiographic images, or the like) and an output (e.g., vessel fiducials).

The ML system 802 may make use of experimental data 808 gathered during several prior procedures. Experimental data 808 can include IVUS images from several IVUS “runs” for several patients. The experimental data 808 may be collocated with the ML system 802 (e.g., stored in a storage 810 of the ML system 802), may be remote from the ML system 802 and accessed via a network interface 804, or may be a combination of local and remote data. As noted above, ML system 802 could be configured to learn relationships between other inputs than IVUS images. In such an example, experimental data 808 would include examples of these inputs. As a specific example contemplated herein, ML system 802 could be configured to learn relationships between angiographic images and vessel fiducials and experimental data 808 would include angiographic images from several angiographic procedures for several patients. It is to be appreciated that although the balance of the description focuses on the depicted example where experimental data 808 includes IVUS images, other images could be used in place of IVUS images.

Experimental data 808 can be used to form training data 812. As noted above, the ML system 802 may include a storage 810, which may include a hard drive, solid state storage, and/or random access memory. The storage 810 may hold training data 812. In general, training data 812 can include information elements or data structures comprising indications of multiple IVUS image series and corresponding desired output (e.g., vessel fiducials). For example, where ML model 822 is to be trained and deployed as one of ML model(s) 128 to infer vessel fiducials 126b from IVUS image series 124, the input can be multiple pairs of IVUS image series and associated vessel fiducials.

The training data 812 may be applied to train ML model 822. Depending on the application, different types of models may be used to form the basis of ML model 822. For instance, in the present example, an artificial neural network (ANN) may be particularly well-suited to learning associations between IVUS image frames (e.g., IVUS image series 124, IVUS image frames 302a, etc.) and vessel fiducials (e.g., vessel fiducials 126b, vessel fiducials 502a, etc.) Convoluted neural networks may also be well-suited to this task. Any suitable training algorithm 818 may be used to train the ML model 822. Nonetheless, the example depicted in FIG. 8 may be particularly well-suited to a supervised training algorithm or reinforcement learning training algorithm. For a supervised training algorithm, the ML system 802 may apply the IVUS image series 814 as inputs 828, to which an expected output (e.g., vessel fiducials 816) can be generated by ML model 822. In a reinforcement learning scenario, training algorithm 818 may attempt to maximize some or all (or a weighted combination) of the model inputs 828 mappings to output 824 to produce an ML model 822 having the least error. With some embodiments, training data 812 can be split into “training” and “testing” data wherein some subset of the training data 812 can be used to adjust the ML model 822 (e.g., internal weights of the model, or the like) while another, non-overlapping subset of the training data 812 can be used to measure an accuracy of the ML model 822 to infer (or generalize) output 824 from “unseen” input 828.

The ML model 822 may be applied using a processor circuit 806, which may include suitable hardware processing resources that operate on the logic and structures in the storage 810. The training algorithm 818 and/or the development of the trained ML model 822 may be at least partially dependent on hyperparameters 820. For example, where ML model 822 is an artificial neural network (ANN), hyperparameters 820 could be the number of hidden layers, nodes in each hidden layer, the activation function, node connection weight initialization, or the like. In exemplary embodiments, the model hyperparameters 820 may be automatically selected based on logic 826, which may include any known hyperparameter optimization techniques as appropriate to the ML model 822 selected and the training algorithm 818 to be used. Again, using the example where ML model is an ANN, logic 826 could comprise a grid search, a random search, or a Bayesian Optimization and could be configured to identify hyperparameters 820 based on these search and/or optimization methods. In optional, embodiments, the ML model 822 may be re-trained over time, to accommodate new knowledge and/or updated experimental data 808.

Once the ML model 822 is trained, it may be applied (e.g., by the processor 108, or the like) to new input data (e.g., IVUS image series 124, etc.) This input to the ML model 822 may be formatted according to a predefined model inputs 828 mirroring the way that the training data 812 was provided to the ML model 822. The ML model 822 may generate output 824 which may be, for example, vessel fiducials 126b as discussed above.

The above description pertains to a particular kind of ML system 802, which applies supervised learning techniques given available training data with input/output pairs. However, the present invention is not limited to use with a specific ML paradigm, and other types of ML techniques may be used. For example, in some embodiments the ML system 802 may apply for example, evolutionary algorithms, or other types of ML algorithms and models to an angiogram image 122 and/or IVUS image series 124 and vessel fiducials 126a and/or vessel fiducials 126b as contemplated herein.

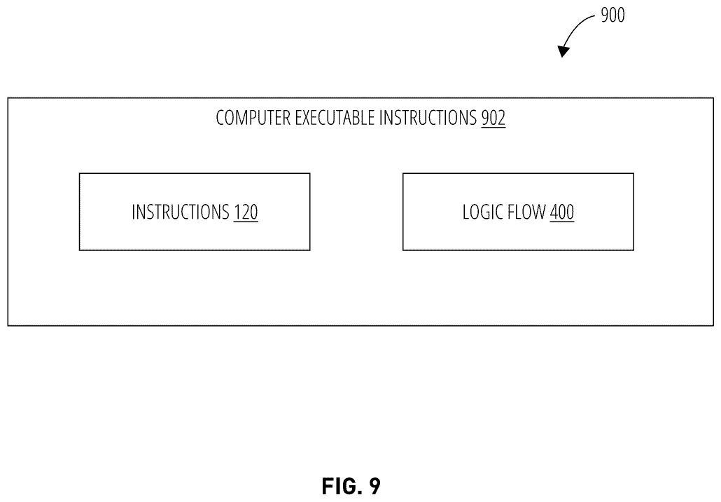

FIG. 9 illustrates computer-readable storage medium 900. Computer-readable storage medium 900 may comprise any non-transitory computer-readable storage medium or machine-readable storage medium, such as an optical, magnetic or semiconductor storage medium. In various embodiments, computer-readable storage medium 900 may comprise an article of manufacture. In some embodiments, computer-readable storage medium 900 may store computer executable instructions 902 with which circuitry (e.g., processor 108, and the like) can execute. For example, computer executable instructions 902 can include instructions to implement operations described with respect to instructions 120 and/or logic flow 400. Examples of computer-readable storage medium 900 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 902 may include any suitable type of code, such as source code, compiled code, interpreted code, executable code, static code, dynamic code, object-oriented code, visual code, and the like.

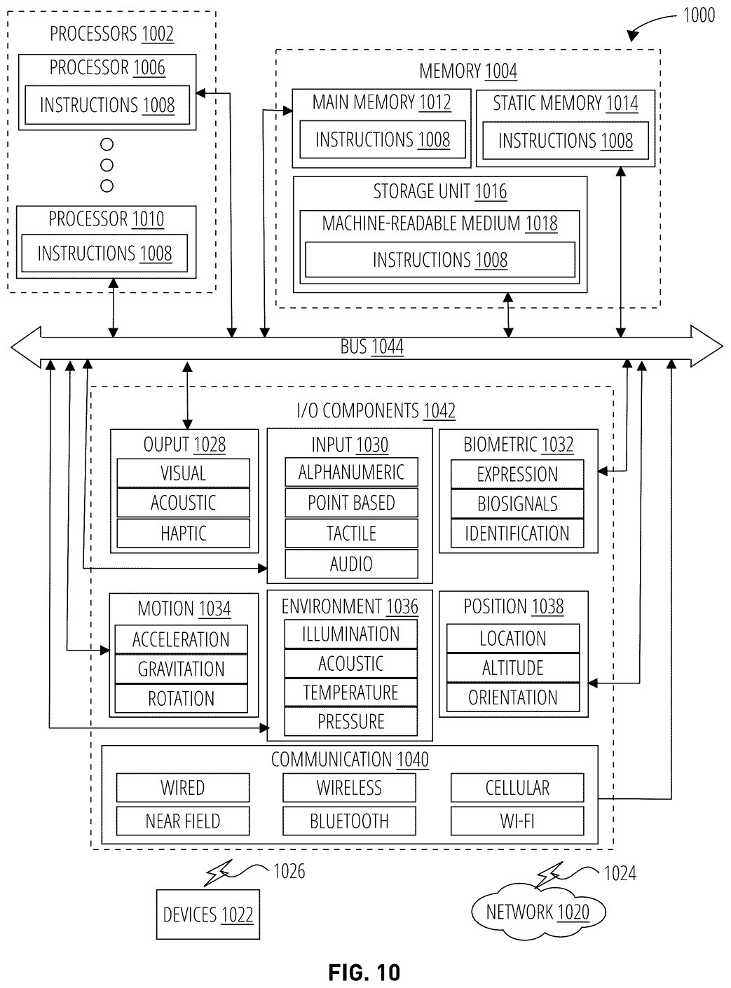

FIG. 10 illustrates a diagrammatic representation of a machine 1000 in the form of a computer system within which a set of instructions may be executed for causing the machine to perform any one or more of the methodologies discussed herein. More specifically, FIG. 10 shows a diagrammatic representation of the machine 1000 in the example form of a computer system, within which instructions 1008 (e.g., software, a program, an application, an applet, an app, or other executable code) for causing the machine 1000 to perform any one or more of the methodologies discussed herein may be executed. For example, the instructions 1008 may cause the machine 1000 to execute logic flow 400 of FIG. 4, or the like. More generally, the instructions 1008 may cause the machine 1000 to automatically align frames in a series of intravascular images (e.g., frames of IVUS image series 124) with the viewing perspective of an extravascular image (e.g., angiogram image 122) by longitudinally and angularly aligning the frames based on the location and angle of vessel fiducials identified from both image modalities.

The instructions 1008 transform the general, non-programmed machine 1000 into a particular machine 1000 programmed to carry out the described and illustrated functions in a specific manner. In alternative embodiments, the machine 1000 operates as a standalone device or may be coupled (e.g., networked) to other machines. In a networked deployment, the machine 1000 may operate in the capacity of a server machine or a client machine in a server-client network environment, or as a peer machine in a peer-to-peer (or distributed) network environment. The machine 1000 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1008, sequentially or otherwise, that specify actions to be taken by the machine 1000. Further, while only a single machine 1000 is illustrated, the term “machine” shall also be taken to include a collection of machines 1000 that individually or jointly execute the instructions 1008 to perform any one or more of the methodologies discussed herein.

The machine 1000 may include processors 1002, memory 1004, and I/O components 1042, which may be configured to communicate with each other such as via a bus 1044. In an example embodiment, the processors 1002 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1006 and a processor 1010 that may execute the instructions 1008. The term “processor” is intended to include multi-core processors that may comprise two or more independent processors (sometimes referred to as “cores”) that may execute instructions contemporaneously. Although FIG. 10 shows multiple processors 1002, the machine 1000 may include a single processor with a single core, a single processor with multiple cores (e.g., a multi-core processor), multiple processors with a single core, multiple processors with multiples cores, or any combination thereof.

The memory 1004 may include a main memory 1012, a static memory 1014, and a storage unit 1016, both accessible to the processors 1002 such as via the bus 1044. The main memory 1004, the static memory 1014, and storage unit 1016 store the instructions 1008 embodying any one or more of the methodologies or functions described herein. The instructions 1008 may also reside, completely or partially, within the main memory 1012, within the static memory 1014, within machine-readable medium 1018 within the storage unit 1016, within at least one of the processors 1002 (e.g., within the processor's cache memory), or any suitable combination thereof, during execution thereof by the machine 1000.

The I/O components 1042 may include a wide variety of components to receive input, provide output, produce output, transmit information, exchange information, capture measurements, and so on. The specific I/O components 1042 that are included in a particular machine will depend on the type of machine. For example, portable machines such as mobile phones will likely include a touch input device or other such input mechanisms, while a headless server machine will likely not include such a touch input device. It will be appreciated that the I/O components 1042 may include many other components that are not shown in FIG. 10. The I/O components 1042 are grouped according to functionality merely for simplifying the following discussion and the grouping is in no way limiting. In various example embodiments, the I/O components 1042 may include output components 1028 and input components 1030. The output components 1028 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1030 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1042 may include biometric components 1032, motion components 1034, environmental components 1036, or position components 1038, among a wide array of other components. For example, the biometric components 1032 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1034 may include acceleration sensor components (e.g., accelerometer), gravitation sensor components, rotation sensor components (e.g., gyroscope), and so forth. The environmental components 1036 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1038 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1042 may include communication components 1040 operable to couple the machine 1000 to a network 1020 or devices 1022 via a coupling 1024 and a coupling 1026, respectively. For example, the communication components 1040 may include a network interface component or another suitable device to interface with the network 1020. In further examples, the communication components 1040 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1022 may be another machine or any of a wide variety of peripheral devices (e.g., a peripheral device coupled via a USB).

Moreover, the communication components 1040 may detect identifiers or include components operable to detect identifiers. For example, the communication components 1040 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1040, such as location via Internet Protocol (IP) geolocation, location via Wi-Fi® signal triangulation, location via detecting an NFC beacon signal that may indicate a particular location, and so forth.

The various memories (i.e., memory 1004, main memory 1012, static memory 1014, and/or memory of the processors 1002) and/or storage unit 1016 may store one or more sets of instructions and data structures (e.g., software) embodying or utilized by any one or more of the methodologies or functions described herein. These instructions (e.g., the instructions 1008), when executed by processors 1002, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1020 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1020 or a portion of the network 1020 may include a wireless or cellular network, and the coupling 1024 may be a Code Division Multiple Access (CDMA) connection, a Global System for Mobile communications (GSM) connection, or another type of cellular or wireless coupling. In this example, the coupling 1024 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1008 may be transmitted or received over the network 1020 using a transmission medium via a network interface device (e.g., a network interface component included in the communication components 1040) and utilizing any one of several well-known transfer protocols (e.g., hypertext transfer protocol (HTTP)). Similarly, the instructions 1008 may be transmitted or received using a transmission medium via the coupling 1026 (e.g., a peer-to-peer coupling) to the devices 1022. The terms “transmission medium” and “signal medium” mean the same thing and may be used interchangeably in this disclosure. The terms “transmission medium” and “signal medium” shall be taken to include any intangible medium that can store, encoding, or carrying the instructions 1008 for execution by the machine 1000, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A complementary image modality correlation and visualization system, comprising:
a processor; and
memory comprising instructions executable by the processor, which when executed cause the system to:
receive an extravascular image of a vessel of a patient;
receive a series of intravascular images of the vessel of the patient, the series of intravascular images comprising a plurality of frames;
identify a plurality of vessel fiducials represented in the extravascular image and the series of intravascular images;
determine an angular offset for at least one or more of the plurality of frames based in part on an angle of orientation of the plurality of vessel fiducials in the extravascular image and the series of intravascular images; and
generate an aligned series of intravascular images comprising the plurality of frames,
wherein the one or more of the plurality of frames are rotated based on the angular offset in the aligned series of intravascular images.

2. The complementary image modality correlation and visualization system of claim 1, the instructions when executed by the processor further cause the system to generate a graphical user interface (GUI), the GUI comprising visual depictions of the extravascular image and the aligned series of intravascular images.

3. The complementary image modality correlation and visualization system of claim 1, the instructions when executed by the processor further cause the system to execute a machine learning (ML) model to infer the plurality of vessel fiducials from the extravascular image.

4. The complementary image modality correlation and visualization system of claim 3, wherein the ML model is trained to infer locations and angle of orientation of the plurality of vessel fiducials from extravascular images.

5. The complementary image modality correlation and visualization system of claim 3, wherein the ML model is a first ML model, and wherein the instructions when executed by the processor further cause the system to execute a second ML model to infer frames of the series of intravascular images comprising the plurality of vessel fiducials from the series of intravascular images.

6. The complementary image modality correlation and visualization system of claim 5, wherein the second ML model is trained to infer angle of orientation of vessel fiducials from a series of intravascular images.

7. The complementary image modality correlation and visualization system of claim 1, wherein the plurality of vessel fiducials comprises a lumen geometry, a vessel geometry, a side branch location, a calcium morphology, a plaque distribution, a guide catheter, a thrombus, and/or a myocardium.

8. The complementary image modality correlation and visualization system of claim 1, the instructions when executed by the processor further cause the system to:
determine a mapping between the plurality of vessel fiducials represented in the extravascular image and the series of intravascular images; and
determine, using the mapping between the plurality of vessel fiducials, a longitudinal offset for at least one of the one or more of the plurality of frames based in part on a location of the plurality of vessel fiducials in the extravascular image and the series of intravascular images,
wherein the at least one of the one or more of the plurality of frames is shifted longitudinally based on the longitudinal offset in the aligned series of intravascular images, and.

9. The complementary image modality correlation and visualization system of claim 8, the instructions when executed by the processor further cause the system to:
determine a first angle, the first angle corresponding to an angle of orientation of a one of the plurality of vessel fiducials represented in the one or more of the plurality of frames;
determine a second angle corresponding to an angle of orientation of the one of the plurality of vessel fiducials represented in the extravascular image; and
derive an offset between the first angle and the second angle.

10. The complementary image modality correlation and visualization system of claim 9, the instructions when executed by the processor further cause the system to:
co-register the plurality of frames of the series of intravascular images with the extravascular image based in part on the mapping between the plurality of vessel fiducials; and
rotate the co-registered plurality of frames of the series of intravascular images based in part on the derived offset between the first angle and the second angle.

11. The complementary image modality correlation and visualization system of claim 9, the instructions when executed by the processor further cause the system to generate a curve comprising indications of the longitudinal offset and/or the angular offset for the plurality of frames based on a line fitting algorithm applied to the longitudinal offset and/or the angular offset.

12. The complementary image modality correlation and visualization system of claim 1, wherein the series of intravascular images are intravascular ultrasound (IVUS) images or optical coherence tomography (OCT) images.

13. The complementary image modality correlation and visualization system of claim 1, wherein the extravascular image is an angiographic image, a computed tomography (CT) image, or a magnetic resonance image (MRI).

14. At least one non-transitory machine readable storage device, comprising a plurality of instructions that in response to being executed by a processor of a complementary image modality correlation and visualization system cause the processor to:
receive an extravascular image of a vessel of a patient;
receive a series of intravascular images of the vessel of the patient, the series of intravascular images comprising a plurality of frames;
identify a plurality of vessel fiducials represented in the extravascular image and the series of intravascular images;
determine an angular offset for at least one or more of the plurality of frames based in part on an angle of orientation of the plurality of vessel fiducials in the extravascular image and the series of intravascular images; and
generate an aligned series of intravascular images comprising the plurality of frames,
wherein the one or more of the plurality of frames are rotated based on the angular offset in the aligned series of intravascular images.

15. The non-transitory machine readable storage device of claim 14, the instructions when executed by the processor further cause the processor to generate a graphical user interface (GUI), the GUI comprising visual depictions of the extravascular image and the aligned series of intravascular images.

16. The non-transitory machine readable storage device of claim 14, the instructions when executed by the processor further cause the processor to:
execute a first machine learning (ML) model to infer the plurality of vessel fiducials from the extravascular image; and
execute a second ML model to infer frames of the series of intravascular images comprising the plurality of vessel fiducials from the series of intravascular images.

17. The non-transitory machine readable storage device of claim 16, the instructions when executed by the processor further cause the processor to:
determine a longitudinal offset for at least a first one of the plurality of frames based in part on a location of the plurality of vessel fiducials in the extravascular image and the series of intravascular images,
wherein the first one of the plurality of frames is shifted longitudinally based on the longitudinal offset in the aligned series of intravascular images, and
wherein the first ML model is trained to infer locations and angle of orientation of the plurality of vessel fiducials from extravascular images, and wherein the second ML model is trained to infer an angle of orientation of vessel fiducials from a series of intravascular images.

18. A method for a complementary image modality correlation and visualization system, comprising:
receiving, by a processor, an extravascular image of a vessel of a patient;
receiving, by the processor, a series of intravascular images of the vessel of the patient, the series of intravascular images comprising a plurality of frames;
identifying, by the processor, a plurality of vessel fiducials represented in the extravascular image and the series of intravascular images;
determining, by the processor, an angular offset for at least one or more of the plurality of frames based in part on an angle of orientation of the plurality of vessel fiducials in the extravascular image and the series of intravascular images;
generating, by the processor, an aligned series of intravascular images comprising the plurality of frames; and
generating, by the processor, a graphical user interface (GUI), the GUI comprising visual depictions of the extravascular image and the aligned series of intravascular images,
wherein the one or more of the plurality of frames is rotated based on the angular offset in the aligned series of intravascular images.

19. The method of claim 18, wherein:
determining, by the processor, a mapping between the plurality of vessel fiducials represented in the extravascular image and the series of intravascular images;
determining, by the processor, a longitudinal offset for at least one of the one or more of the plurality of frames based in part on a location of the plurality of vessel fiducials in the extravascular image and the series of intravascular images; and
determining the angular offset for at least the one or more of the plurality of frames based in part on the angle of orientation of the plurality of vessel fiducials in the extravascular image and the series of intravascular images comprises:
determining, by the processor, a first angle, the first angle corresponding to an angle of orientation of a one of the plurality of vessel fiducials represented in the one or more of the plurality of frames;
determining, by the processor, a second angle corresponding to an angle of orientation of the one of the plurality of vessel fiducials represented in the extravascular image; and
deriving, by the processor, an offset between the first angle and the second angle,
wherein the first one of the plurality of frames is shifted longitudinally based on the longitudinal offset in the aligned series of intravascular images, and.

20. The method of claim 18, wherein the plurality of vessel fiducials comprises a lumen geometry, a vessel geometry, a side branch location, a calcium morphology, a plaque distribution, a guide catheter, a thrombus, and/or a myocardium.
